(19) United States
(12) Patent Application Publication
(10) Pub. No.: US 2026/0183286 A1
(43) Pub. Date: Jul. 2, 2026

(54) Compositions and Methods for an Oral Supplement to Oral Minoxidil for the Treatment of Alopecia

(71) Applicant: FOLLEA INTERNATIONAL, Irvine, CA (US)

(72) Inventor: Ofer A. Goren, Prague (CZ)

(21) Appl. No.: 18/552,401

(22) PCT Filed: Sep. 1, 2023
(86) PCT No.: PCT/US2023/031806

§ 371 Date: Sep. 25, 2023

(51) Int. Cl. A61K 31/506; A61K 9/00; A61K 33/20; A61K 36/14; A61K 36/484; A61K 36/82

(52) U.S. Cl. CPC A61K 31/506; A61K 9/0053; A61K 33/20; A61K 36/14; A61K 36/484; A61K 36/82

(57) ABSTRACT

Embodiments can relate to compositions and methods for the treatment of hair loss, which can include treatment of alopecia. The compositions and methods relate to an oral supplement to oral minoxidil includes a SLC22A9 inducer, a HIF-alpha inducer, and/or an acidifying agent. Acidifying blood of a patient suffering from alopecia can cause mild acidosis which can up-regulate SLC22A9 and/or down-regulate AABC3. Inducing SLC22A9, inducing HIF-alpha, and/or acidifying blood of the patient can facilitate oral forms of minoxidil to enter hair follicles.

### FIELD OF THE INVENTION

Embodiments can relate to compositions and methods for the treatment of hair loss, which can include treatment of alopecia. Specifically, the compositions and methods relate to an oral supplement to oral minoxidil.

### BACKGROUND OF THE INVENTION

A drug that can be used for the treatment of alopecia is minoxidil. In order for minoxidil to exert its hair growth properties, minoxidil must be activated by the SULT1A1 (minoxidil sulfotransferase) enzyme. The enzyme is expressed both in the scalp and the liver. Different people express different amount of the enzyme or possess genetic variations that effect the activity of the enzyme; thus, the response to minoxidil treatment is variable among people and dependent on the SULT1A1 enzyme.

Previous research demonstrated that alkalinizing the intracellular pH of keratinocytes induces the expression of SULT1A1 enzyme in the scalp and subsequently improves, increases, or enhances minoxidil response. Accordingly, a topical alkalizing agent was developed as a minoxidil booster; however, this topical minoxidil booster was met with limited success since it is difficult to comply with twice daily applications, it tends to change hair color, and it tends to leave residue from sodium bicarbonate on the hair. Further, minoxidil exerts its hair re-growth effect mainly through the dermal papillae and dermal sheath cells which are difficult to treat with topical cosmetic products. The dermal papillae have a rich blood supply, which can make it easier to modulate with orally administered compositions (e.g., drugs, ingredients, supplements, etc.). As such, an oral composition that increases SULT1A1 enzyme in dermal papillae, dermal sheath, or outer root sheath of hair follicles and subsequently increases the response to minoxidil for the treatment of hair loss would be advantageous.

Sulfation of oral minoxidil occurs in the liver; not at the hair follicle. Thus, oral minoxidil does not enter the hair follicle. As will be explained in the present disclosure, the inventor has discovered that expression of the SLC22A9 can facilitate oral minoxidil entering the hair follicle.

### SUMMARY OF THE INVENTION

Embodiments relate to compositions and methods related to an oral supplement to oral minoxidil. As noted above, sulfation of oral minoxidil occurs in the liver; not at the hair follicle, which leads to oral minoxidil not entering the hair follicle. Expression of the SLC22A9, however, can facilitate oral minoxidil entering the hair follicle. Expression of SLC22A9 can be achieved by inducing SLC22A9, HIF-1-A, and/or acidifying the blood of the patient.

An exemplary embodiment relates to an oral supplement to oral minoxidil. The oral supplement can include a SLC22A9 inducer, a HIF-alpha inducer, and/or an acidifying agent.

In some embodiments, the acidifying agent can up-regulate SLC22A9 and/or down-regulate AABC3.

In some embodiments, the HIF-alpha inducer can include Juniperus cummunis Extract, Green Tree Extract, Glycyrrhiza glabra, o-phenanthroline, iodochlorohydroxyquinoline, and/or cobalt sulfate heptahydrate.

In some embodiments, the oral supplement can include an oral minoxidil treatment composition, the oral minoxidil treatment composition including minoxidil or a pharmaceutically acceptable salt thereof.

In some embodiments, the minoxidil or pharmaceutically acceptable salt thereof can be present in the oral minoxidil treatment composition at a dosage within a range from 0.125 mg to 10 mg.

In some embodiments, the acidifying agent can include ammonium chloride, methanol, salicylate, biguanide, alcohol, polyhydric sugar, cyanide, and/or carbon monoxide.

An exemplary embodiment relates to a method for treating alopecia. The method can involve treating for alopecia by: administering oral minoxidil to a patient suffering from alopecia; and administering an oral supplement before, during, and/or after administration of oral minoxidil, the oral supplement including a SLC22A9 inducer, a HIF-alpha inducer, and/or an agent to acidify blood of the patient. Alternatively, the method can involve treating for alopecia by administering an oral supplement containing oral minoxidil, the oral supplement further including a SLC22A9 inducer, a HIF-alpha inducer, and/or an agent to acidify blood of the patient.

In some embodiments, the acidifying agent can up-regulate SLC22A9 and/or down-regulate AABC3.

In some embodiments, the acidifying agent can cause mild acidosis for the patient.

In some embodiments, the HIF-alpha inducer can include Juniperus cummunis Extract, Green Tree Extract, Glycyrrhiza glabra, o-phenanthroline, iodochlorohydroxyquinoline, and/or cobalt sulfate heptahydrate.

In some embodiments, the oral minoxidil can include minoxidil or a pharmaceutically acceptable salt thereof.

In some embodiments, the minoxidil or pharmaceutically acceptable salt thereof can be present in the oral minoxidil at a dosage within a range from 0.125 mg to 10 mg.

In some embodiments, the acidifying agent can include ammonium chloride, methanol, salicylate, biguanide, alcohol, polyhydric sugar, cyanide, and/or carbon monoxide.

An exemplary embodiment relates to an oral composition for treating alopecia. The composition can include a SLC22A9 inducer, a HIF-alpha inducer, and/or an acidifying agent.

In some embodiment, the acidifying agent can up-regulate SLC22A9 and/or down-regulate AABC3.

In some embodiment, the HIF-alpha inducer can include Juniperus cummunis Extract, Green Tree Extract, Glycyrrhiza glabra, o-phenanthroline, iodochlorohydroxyquinoline, and/or cobalt sulfate heptahydrate.

In some embodiment, the acidifying agent can include ammonium chloride, methanol, salicylate, biguanide, alcohol, polyhydric sugar, cyanide, and/or carbon monoxide.

An exemplary embodiment relates to a method for treating alopecia. The method involves administering an oral composition to a patient suffering from alopecia, the oral composition comprising a SLC22A9 inducer, a HIF-alpha inducer, and/or an acidifying agent.

In some embodiment, the acidifying agent can up-regulate SLC22A9 and/or down-regulate AABC3.

In some embodiment, the HIF-alpha inducer includes Juniperus cummunis Extract, Green Tree Extract, Glycyrrhiza glabra, o-phenanthroline, iodochlorohydroxyquinoline, and/or cobalt sulfate heptahydrate.

In some embodiment, the acidifying agent can include ammonium chloride, methanol, salicylate, biguanide, alcohol, polyhydric sugar, cyanide, and/or carbon monoxide.

As used herein, the terms “prevent” or “prevention” and other derivatives of the words, when used in reference to alopecia, e.g., androgenetic alopecia, can refer to a reduced likelihood of alopecia in an individual receiving a given treatment relative to that of a similar individual at risk for alopecia but not receiving that treatment. As such, the terms “prevent” and “prevention” can encompass a treatment that results in a lesser degree of alopecia than would be otherwise expected for a given individual. Efficacy for prevention of alopecia can be established through controlled studies, e.g., in which a subject is administered a treatment (e.g., a topical, oral, transdermal, etc.) and another subject is administered a placebo. Under these circumstances, if the subject treated with the treatment undergoes less hair loss over time relative to the subject receiving the placebo, e.g., at least 5% less, at least 10% less, at least 15% less, at least 20% less, at least 25% less, at least 30% less, at least 35% less, at least 40% less, at least 45% less, at least 50% less or beyond, the treatment can be considered effective for the prevention of alopecia.

As used herein, the term “subject” refers to a human or animal in need of a therapeutic treatment for androgenetic alopecia or any other form of alopecia.

As used herein, the terms “treat,” “treatment,” or “treating” refer to therapeutic treatments, wherein the object is to reverse, alleviate, ameliorate, inhibit, slow down or stop the progression or severity of a disease or condition, e.g., androgenetic alopecia or other form of alopecia. The term “treating” can include reducing or alleviating at least one adverse effect or symptom of the disease or condition. Treatment can be considered to be generally “effective” if one or more symptoms are reduced. Alternatively, treatment can be “effective” if the progression of a disease is reduced or halted. That is, “treatment” can include not just the improvement of symptoms, but also a cessation of, or at least slowing of, progress or worsening of symptoms compared to what would be expected in the absence of treatment. Beneficial or desired clinical results can include, but are not limited to, alleviation of one or more symptom(s), diminishment of extent of disease, stabilized (e.g., not worsening) state of disease, delay or slowing of disease progression, amelioration or palliation of the disease state, remission (whether partial or total), and/or decreased mortality. For example, treatment can be considered effective if the extent or amount of hair loss is reduced, or the progression of hair loss is slowed or halted. The term “treatment” of a disease can also include providing relief from the symptoms or side-effects of the disease (including palliative treatment).

Efficacy of treatment to treat or prevent alopecia can be determined by monitoring the density of hairs on a given area of the subject's body, e.g., a given area of the scalp. For example, if the rate of hair loss is reduced, e.g., by 10% or more following treatment, the treatment can be considered effective for the prevention of alopecia. Similarly, if hair density remains the same, the treatment can be considered to be effective for the prevention of alopecia. If the density of hair increases, e.g., by 5% or more, e.g., by 10% or more following treatment, the treatment can also be considered effective for the treatment and/or prevention of androgenetic alopecia. Efficacy of treatment to treat or prevent androgenetic alopecia can be determined by monitoring global photography. For example, the patient or an expert can assess the treatment response utilizing before and after global photographs.

As used herein the term “alopecia” refers to all forms of hair loss in humans or animals, both male and female, including but not limited to traction alopecia, androgenetic alopecia, male pattern baldness, female pattern hair loss, alopecia areata, alopecia universalis, telogen effluvium, chemotherapy induced alopecia, hair shedding, eyebrow hair loss, beard hair loss, hair thinning, etc. The term permits the presence of elements that do not materially affect the basic and novel or functional characteristic(s) of that embodiment.

To utilize the compositions described herein, subject can apply an effective amount of the composition to the scalp. The term “effective amount,” as used herein, can be an amount which is effective in preventing or treating hair loss.

Further features, aspects, objects, advantages, and possible applications of the present invention will become apparent from a study of the exemplary embodiments and examples described below, in combination with the Figures, and the appended claims.

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

Alopecia, and in particular androgenetic alopecia, is the progressive miniaturization of hair follicles on the scalp. It can be characterized by diffuse thinning of the crown region of the scalp while the frontal hairline remains intact. Alopecia is generally hereditary, but can also be dependent on hormones that may influence its development. Oral and/or topical minoxidil is an U.S. FDA approved drug for the treatment of alopecia. Its use can provide a moderate increase in hair regrowth. The risk for adverse events with such minoxidil use is low; however, irritant dermatitis, allergic contact dermatitis, and hypertrichosis have been reported.

While embodiments disclosed herein may specifically discuss treatment of a form of alopecia (e.g., androgenetic alopecia), it is understood that the compositions and methods disclosed can be similarly applicable for treatment of other forms of alopecia.

Embodiments can relate to a method for treating alopecia. The method can involve treating for alopecia by administering oral minoxidil to a patient suffering from alopecia. The method can involve administering an oral supplement before, during, and/or after administration of oral minoxidil. The oral supplement can include a SLC22A9 inducer, a HIF-1-A inducer, and/or an agent to acidify blood of the patient.

Embodiments can relate to compositions for treating alopecia. For example, embodiments can relate to compositions pertaining to an oral supplement to oral minoxidil. The composition can be administered to induce the solute carrier family 22 (e.g., SLC2A9), induce the hypoxia-inducible factor 1-alpha (e.g., HIF-1-A), and/or acidify blood of the patient (e.g., using ammonium chloride) suffering from alopecia. Acidifying blood of the patient can up-regulate SLC22A9 and/or down-regulate AABC3. Up-regulating SLC22A9 and/or down-regulating AABC3 can allow or facilitate orally administered minoxidil to enter the hair follicle.

The composition is an oral supplement to oral minoxidil. Thus, it is contemplated for the composition to be used in conjunction with oral minoxidil treatment. The minoxidil treatment can be a treatment involving administration of a composition containing minoxidil. The oral supplement can be administered before (e.g., before the minoxidil composition is administered), during (e.g., while the minoxidil composition is administered), and/or after (e.g., after the minoxidil composition is administered) the oral minoxidil is administered. This can include administering the oral supplement and the minoxidil at the same time, each administered on the same day but at different times, etc. The oral supplement can be separate from the oral minoxidil composition (e.g., be a separate oral supplement composition that is taken in conjunction with an oral form of minoxidil), be part of the minoxidil composition (e.g., be a component in the oral form of minoxidil), or the minoxidil can be part of the oral supplement composition (e.g., the oral supplement composition can include minoxidil).

The minoxidil composition or minoxidil ingredient of a composition can be minoxidil or a pharmaceutically acceptable salt thereof. Minoxidil is a pro-drug converted to its active form, minoxidil sulfate, by sulfotransferase enzymes present in the outer root sheath (ORS) of hair follicles (See Buhl A E, Waldon D J, Baker C A, Johnson G A. Minoxidil sulfate is the active metabolite that stimulates hair follicles. J Invest Dermatol. 1990 November; 95 (5):553-7). It has been demonstrated that the activity of sulfotransferase in the ORS determines the clinical response to minoxidil (See Goren A, Castano JA, McCoy J, Bermudez F, Lotti T. Novel enzymatic assay predicts minoxidil response in the treatment of androgenetic alopecia. Dermatol Ther. 2014; 27 (3):171-3). It is contemplated for the minoxidil dosage to be about 0.125 mg to about 10 mg per day. However, other dosages can be used.

With embodiments of the oral supplement composition being used in conjunction with minoxidil, minoxidil can enter the hair follicle. When entering the hair follicle, minoxidil induces (up-regulates) the expression of sulfotransferases in hair bearing skin, hair follicles, and/or keratinocyte cells, e.g., the scalp. This can include up-regulating the sulfonating capacity. Thus, embodiments of the oral supplement composition having minoxidil as a component of the oral supplement composition can induce the expression of sulfotransferases.

The oral supplement composition and/or the minoxidil composition can be administered at a predetermined frequency. This predetermined frequency can be once every 24 hours, twice every 24 hours, three times every 24 hours, four times every 24 hours, etc. The frequency of administration of the oral supplement composition can be the same as or different from the frequency of minoxidil.

The form of the oral supplement composition can be in tablet form, pill form, capsule form, gel form, liquid form, spray form, etc. The form the minoxidil composition can be in tablet form, pill form, capsule form, gel form, liquid form, spray form, etc. The form of the oral supplement composition can be the same as or different from that of the minoxidil composition.

Any of the formulations of the oral supplement composition and/or that of the minoxidil composition can include a carrier. For instance, any of the formulations can be encapsulated in a pharmaceutically acceptable carrier. The pharmaceutically acceptable carrier can encapsulate the composition in liposomes, sodium alginate, gum Arabic, chitosan, cellulose, pectin, etc. In addition to being “pharmaceutically acceptable,” each carrier can be “acceptable” in the sense of being compatible with the other ingredients of the formulation. The carrier can be used to temporarily encapsulate the composition until a condition is met that causes its release. In some embodiments, the carrier can be formulated to controllably release the composition—e.g., a time-dependent release.

An exemplary embodiment of the oral supplement composition can be formulated as an oral supplement to oral minoxidil. For instance, the oral supplement can be administered in conjunction with oral minoxidil treatment—e.g., a patient suffering from alopecia can take composition(s) of the oral supplement before, during, and/or after taking an oral minoxidil composition. The oral supplement can include an agent configured to express SLC22A9 so as to up-regulate SLC22A9. Thus, the oral supplement can include a SLC22A9 inducer as the agent. In addition, or in the alternative, the oral supplement can include an agent configured to express HIF-1-A so as to up-regulate HIF-1-A. Thus, the oral supplement can include a HIF-1-A inducer as the agent. Examples of HIF-1-A inducers can include Juniperus cummunis Extract, Green Tree Extract, Glycyrrhiza glabra, o-phenanthroline, iodochlorohydroxyquinoline, cobalt sulfate heptahydrate, etc. The oral supplement can include an agent configured to acidify blood of the patent so as to up-regulate SLC22A9 and/or down-regulate AABC3. It is contemplated for the blood acidifying agent to acidify the blood so as to induce mild acidosis. An example of a blood acidifying agent can be ammonium chloride. For instance, oral administration of 0.1 g/kg (1.9 mEq/kg) of ammonium chloride can induce metabolic acidosis in a patient. Other acidosis agents can include methanol, salicylates, biguanides, alcohols, polyhydric sugars, cyanide, carbon monoxide, etc. The oral supplement can include a SLC22A9 inducer, a HIF-1-A inducer, and/or an acidifying agent. The oral supplement can include one or more SLC22A9 inducers, one or more HIF-1-A inducers, or one or more acidifying agents.

The oral supplement can include one or more compositions, and may also include an oral minoxidil treatment composition. The oral minoxidil treatment composition can include minoxidil or a pharmaceutically acceptable salt thereof. The minoxidil or pharmaceutically acceptable salt thereof can be present in the oral minoxidil treatment composition at a dosage within a range from 0.125 mg to 10 mg per day. The minoxidil can be included in a composition that is separate from the oral supplement or be part of the oral supplement.

As noted herein, the oral supplement can include a SLC22A9 inducer, a HIF-1-A inducer, and/or an acidifying agent. The oral supplement can be a single composition that includes one or more of a SLC22A9 inducer, a HIF-1-A inducer, or an acidifying agent. The oral supplement can be a two or more compositions—e.g., one composition can include a SLC22A9 inducer or a HIF-1-A inducer, while another composition includes an acidifying agent. The two compositions can be administered to the patient separately but both in conjunction with minoxidil. It is understood that more or less compositions can be used and other combinations of inducers/agents in the compositions can be used.

As another example, the oral supplement composition can include one or more SLC22A9 inducers, one or more HIF-1-A inducers, and/or one or more acidifying agents. For instance, the treatment might involve administering composition-1 and composition-2, wherein composition-1 has a first inducer or acidifying agent that differs from an inducer or acidifying agent used in composition-2, or composition-1 has an inducer or acidifying agent at a first concentration and composition-2 has the same inducer or acidifying agent but at a different concentration, etc. In addition, one composition might be administered before administration of minoxidil, while the other might be administered after administration of minoxidil.

How many compositions to use, how many inducers or acidifying agents to use, when to administer them, etc. can be determined based on desired effects and/or design criteria.

An exemplary formulation of an embodiment of the oral supplement composition includes the following:

| | | |
|---|---|---|
| Juniperus | (natural levels of Hinokitiol) | 500 mg |
| cummunis Extract | | |
| Green Tree | (natural levels of Epicatechin-3-gallate) | 400 mg |
| Extract | | |
| Glycyrrhiza glabra | (natural levels of Dibenzoylmethane) | 400 mg |
| (liquorice root | | |
| extract) | | |
| Chloride | (as Ammonium Chloride) | 268 mg |
| Ammonium | (as Ammonium Chloride) | 132 mg |
| Potassium | | 4 mg |
| Phosphate | | |

A placebo controlled study was conducted with a 268 mg ammonium chloride oral supplement to ascertain increase in SLC22A9 up-regulation. Ten subjects were administered the placebo and 10 subjects were administered ammonium chloride daily for 5 days. The SLC22A9 expression from hair follicles was measured at baseline and day 5. SLC22A9 increased 3 folds and was statistically significant p=0.01 increased in the active vs placebo group.

Embodiments of the oral supplement composition can include one or more sulfotransferase enzymes. Any of the sulfotransferase enzymes can be human or non-human. Examples of a sulfotransferase enzyme can be a carbohydrate sulfotransferase (e.g., CHST1, CHST2, CHST3, CHST4, CHST5, CHST6, CHST7, CHST8, CHST9, CHST10, CHST11, CHST12, CHST13, CHST14, etc.), a galactose-3-O-sulfotransferase (e.g., GAL3ST1, GAL3ST2, GAL3ST3, GAL3ST4, etc.), a heparan sulfate 2-O-sulfotransferase (e.g., HS2ST1), a heparan sulfate 3-O-sulfotransferase (e.g., HS3ST1, HS3ST2, HS3ST3A1, HS3ST3B1, HS3ST4, HS3ST5, HS3ST6, etc.), a heparan sulfate 6-O-sulfotransferase (e.g., HS6ST1, HS6ST2, HS6ST3, etc.), a N-deacetylase/N-sulfotransferase (e.g., NDST1, NDST2, NDST3, NDST4, etc.), a tyrosylprotein sulfotransferase (e.g., TPST1, TPST2, etc.), an uronyl-2-sulfotransferase, an estrone sulfotransferase, a chondroitin 4-sulfotransferase, SULT1A1, SULT1A2, SULT1A3, SULT1A4, SULT1B1, SULT1C2, SULT1C3, SULT1C4, SULT1D1P, SULT1E1, SULT2A1, SULT2B1, SULT4A1, SULT6B1, etc.

The sulfotransferase enzyme can be a phenol sulfotransferase extract (e.g., phenol sulfotransferase extracted from a plant producing a sulfated salicinoid). Examples of plants producing sulfated salicinoids can include Populus trichocarpa, Populus x canescens, Salix sp., Arabidopsis thaliana, silver poplar (Populus alba), Simon's poplar (Populus simonii), S. purpurea, P. tremuloides, I. polycarpa, P. nigra, Oryza sativa, Salicaceae family, Poplars (Populus sp.), Idesia polycarpa-Japanese orange cherry, Potato (Solanum tuberosum), Cabbage (Brassica rapa), Populus trichocarpa (Black cottonwood), Salix Sp. (Willow tree), Cotton (Gossypium), Olea europaea L, Brassica napus, Apium graveolens, Brassica oleracea, Saccharina japonica, Solanum tuberosum, Black cottonwood, etc.

The sulfotransferase enzyme can be OeST1 (olive tree SULT1A1). OeST1 is a sulfotransferase plant supplement that can be extracted from olive trees. The human homolog of OeST1 is SULT1A1, which belongs to the cytosolic sulfotransferase family. Like OeST1, SULT1A1 catalyzes the transfer of a sulfonate group from PAPS to the hydroxyl group of various phenolic substrates, including flavonoids, catecholamines, and xenobiotics. The expression level of OeST1 can vary depending on the tissue, developmental stage, and environmental conditions of the olive plant. However, studies have shown that OeST1 is most abundant in the fruit and leaves of olive. In olive fruit, OeST1 is expressed at high levels during the early stages of development and ripening, when flavonoids and other phenolic compounds are synthesized and accumulated. The expression of OeST1 in olive fruit is regulated by various factors, including light, temperature, and water availability, which can affect the synthesis and accumulation of flavonoids and other phenolics in the fruit. In olive leaves, OeST1 is also expressed at high levels, particularly in young and actively growing leaves. The expression of OeST1 in olive leaves is regulated by various factors, including light, temperature, and biotic and abiotic stresses, which can affect the biosynthesis and accumulation of flavonoids and other phenolics in the leaves. Overall, the expression of OeST1 in olive fruit and leaves suggests that sulfated flavonoids and other phenolics may play important roles in the physiology and adaptation of the olive plant, and may contribute to the health benefits of olive fruit and leaves. There have been studies that measured the concentration of OeST1 (olive tree SULT1A1) in olive leaves. For example, a study published in the Journal of Agricultural and Food Chemistry in 2014 measured the expression levels of different sulfotransferase genes, including OeST1, in olive leaves using quantitative real-time polymerase chain reaction (qPCR) analysis. The study found that OeST1 was expressed at relatively high levels in olive leaves compared to other sulfotransferase genes. Another study published in the same journal in 2018 measured the activity of sulfotransferases, including OeST1, in olive leaf extracts using a radiolabeling assay. The study found that sulfotransferase activity in olive leaf extracts was mainly attributed to OeST1, and that its activity was higher in younger leaves compared to older ones. These studies suggest that OeST1 is present in significant amounts in olive leaves and is likely involved in the sulfation of various phenolic compounds in this plant tissue. However, it is important to note that the concentration of OeST1 or any other enzyme in olive leaves may vary depending on several factors, such as the cultivar, the growth conditions, and the age of the leaves. Therefore, further studies may be necessary to determine the concentration of OeST1 in different types of olive leaves and under different growth conditions.

While some embodiments use the inventive composition as an oral supplement to oral minoxidil, the inventive composition can be used without minoxidil to treat alopecia. For instance, embodiments can relate to an oral composition for treating alopecia, wherein the composition includes a SLC22A9 inducer, a HIF-alpha inducer, and/or an acidifying agent. Thus, hair growth can be promoted, hair loss can be prevented, and/or the rate of hair loss can be reduced by administration of the inventive composition which will induce SLC22A9, induce HIF-alpha, and/or acidify blood of the patient. Again, the acidifying agent can up-regulate SLC22A9 and/or down-regulate AABC3. The acidifying agent can include ammonium chloride, methanol, salicylate, biguanide, alcohol, polyhydric sugar, cyanide, and/or carbon monoxide. The HIF-alpha inducer can include Juniperus cummunis Extract, Green Tree Extract, Glycyrrhiza glabra, o-phenanthroline, iodochlorohydroxyquinoline, and/or cobalt sulfate heptahydrate.

It is understood that any plant the produces flavonoids or has minoxidil sulfotransferase enzymes, phenol sulfotransferase enzymes, and/or sulfotransferase enzymes can be used. Any of the methods and compositions can use the natural concentration of the enzyme found in the plant or an extract thereof. Further, any of the enzymes can be animal derived as well.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the compositions and methods of using and making the same disclosed herein have been discussed and illustrated, it is to be distinctly understood that the invention is not limited thereto but may be otherwise variously embodied and practiced within the scope of the following claims.

What is claimed is:

1. An oral supplement to oral minoxidil, the oral supplement, comprising:
a SLC22A9 inducer, a HIF-alpha inducer, and/or an acidifying agent.

2. The oral supplement of claim 1, wherein:
the acidifying agent up-regulates SLC22A9 and/or down-regulates AABC3.

3. The oral supplement of claim 1, wherein:
the HIF-alpha inducer includes Juniperus cummunis Extract, Green Tree Extract, Glycyrrhiza glabra, o-phenanthroline, iodochlorohydroxyquinoline, and/or cobalt sulfate heptahydrate.

4. The oral supplement of claim 1, further comprising:
an oral minoxidil treatment composition, the oral minoxidil treatment composition including minoxidil or a pharmaceutically acceptable salt thereof.

5. The oral supplement of claim 4, wherein:
the minoxidil or pharmaceutically acceptable salt thereof is present in the oral minoxidil treatment composition at a dosage within a range from 0.125 mg to 10 mg.

6. The oral supplement of claim 1, wherein:
the acidifying agent includes ammonium chloride, methanol, salicylate, biguanide, alcohol, polyhydric sugar, cyanide, and/or carbon monoxide.

7. A method for treating alopecia, the method comprising:
treating for alopecia by:
administering oral minoxidil to a patient suffering from alopecia; and
administering an oral supplement before, during, and/or after administration of oral minoxidil, the oral supplement including a SLC22A9 inducer, a HIF-alpha inducer, and/or an agent to acidify blood of the patient.
or
treating for alopecia by administering an oral supplement containing oral minoxidil, the oral supplement further including a SLC22A9 inducer, a HIF-alpha inducer, and/or an agent to acidify blood of the patient.

8. The method of claim 7, wherein:
the acidifying agent up-regulates SLC22A9 and/or down-regulates AABC3.

9. The method of claim 7, wherein:
the acidifying agent causes mild acidosis for the patient.

10. The method of claim 7, wherein:
the HIF-alpha inducer includes Juniperus cummunis Extract, Green Tree Extract, Glycyrrhiza glabra, o-phenanthroline, iodochlorohydroxyquinoline, and/or cobalt sulfate heptahydrate.

11. The method of claim 7, wherein:
the oral minoxidil includes minoxidil or a pharmaceutically acceptable salt thereof.

12. The method of claim 11, wherein:
the minoxidil or pharmaceutically acceptable salt thereof is present in the oral minoxidil at a concentration within a range from 0.125 mg to 10 mg.

13. The method of claim 7, wherein:
the acidifying agent includes ammonium chloride, methanol, salicylate, biguanide, alcohol, polyhydric sugar, cyanide, and/or carbon monoxide.

14. An oral composition for treating alopecia, the composition, comprising:
a SLC22A9 inducer, a HIF-alpha inducer, and/or an acidifying agent.

15. The oral composition of claim 14, wherein:
the acidifying agent up-regulates SLC22A9 and/or down-regulates AABC3.

16. The oral composition of claim 14, wherein:
the HIF-alpha inducer includes Juniperus cummunis Extract, Green Tree Extract, Glycyrrhiza glabra, o-phenanthroline, iodochlorohydroxyquinoline, and/or cobalt sulfate heptahydrate.

17. The oral composition of claim 14, wherein:
the acidifying agent includes ammonium chloride, methanol, salicylate, biguanide, alcohol, polyhydric sugar, cyanide, and/or carbon monoxide.

18. A method for treating alopecia, the method comprising:
administering an oral composition to a patient suffering from alopecia, the oral composition comprising a SLC22A9 inducer, a HIF-alpha inducer, and/or an acidifying agent.

19. The method of claim 18, wherein:
the acidifying agent up-regulates SLC22A9 and/or down-regulates AABC3.

20. The method of claim 18, wherein:
the HIF-alpha inducer includes Juniperus cummunis Extract, Green Tree Extract, Glycyrrhiza glabra, o-phenanthroline, iodochlorohydroxyquinoline, and/or cobalt sulfate heptahydrate.

21. The method of claim 18, wherein:
the acidifying agent includes ammonium chloride, methanol, salicylate, biguanide, alcohol, polyhydric sugar, cyanide, and/or carbon monoxide.
